(19) United States
(12) Patent Application Publication
(10) Pub. No.: US 2019/0330204 A1
(43) Pub. Date: Oct. 31, 2019

(54) PROCESS FOR THE PREPARATION OF HERBICIDAL COMPOUNDS

(71) Applicant: SYNGENTA PARTICIPATIONS AG, Basel (CH)

(72) Inventors: Rupesh PATRE, Corlim Ilhas (IN); Tomas SMEJKAL, Stein (CH)

(73) Assignee: SYNGENTA PARTICIPATIONS AG, Basel (CH)

(21) Appl. No.: 16/097,107

(22) PCT Filed: Apr. 24, 2017
(86) PCT No.: PCT/EP2017/059613

§ 371 Date: Oct. 26, 2018

(30) Foreign Application Priority Data
Apr. 29, 2016 (IN) 201611015025

(51) Int. Cl. C07D 471/04; C07D 401/04

(52) U.S. Cl. CPC C07D 471/04; C07D 401/04

(57) ABSTRACT

The present invention relates to a process for the preparation of a compound of formula (IX) wherein R¹, R^{2A}, R^{2B}, R³, R⁴, R⁵ and R⁶ are as defined in the specification. Furthermore, the present invention also relates to reduction of the compound of formula (IX) to produce a compound of formula (I).

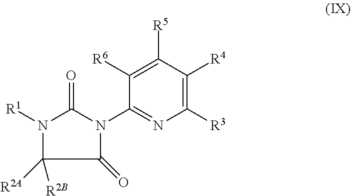

The present invention relates to the preparation of pyridinylimidazolones of formula (IX)

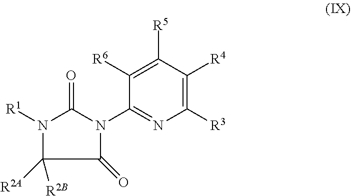

wherein R¹ is selected from C₁-C₆ alkyl, aryl and hydrogen, R^{2A} is selected from C₁-C₆ alkyl and hydrogen, R^{2B} is selected from C₁-C₆ alkyl and hydrogen or R¹ and R^{2A} or R^{2B}, together with the nitrogen and carbon atoms to which they are attached form a 3-7 membered saturated ring optionally comprising from 1 to 3 heteroatoms independently selected from S, O and N and optionally substituted with from 1 to 3 groups independently selected from hydroxyl, ═O, C₁-C₆ alkyl and C₁-C₆ haloalkyl and R³, R⁴, R⁵ and R⁶ are each independently selected from hydrogen, C₁-C₆ alkyl, C₁-C₆ haloalkyl, nitro and halogen. In addition, the present invention relates to the reduction of the compound of formula (IX) to produce a compound of formula (I):

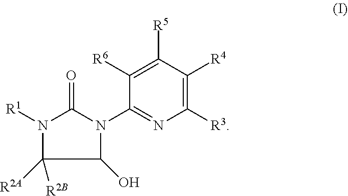

Some pyridinylimidazolones of general formula (I) are known to be herbicidally active as described in WO 2015/059262, WO 2015/052076 and U.S. Pat. No. 4,600,430.

Methods of preparing some pyridinylimidazolones derivatives of formula (I) are described in WO 2015/059262, WO 2015/052076 and U.S. Pat. No. 4,600,430. The present invention offers unique methods to prepare such compounds using less process steps (presenting therefore advantages such as a higher throughput capacity and a lower amount of waste) as well as more attractive conditions (for example providing lower amounts of by-products and avoiding low and high temperature conditions and transition metal catalysts). Further, the present invention is suitable for commercial scale production.

It has been described (WO 2015/052076) that some compounds of formula (I) (R^{2B}=hydrogen) may be prepared by reaction of an amino-pyridine (V) with phenyl chloroformate to give a carbamate product (VI). The subsequent reaction with an appropriately substituted amino-ester (VII) gives compounds of formula (VIII), subsequent cyclisation gives compounds of formula (IX) and reduction with e.g. sodium borohydride gives compounds of formula (I). This process is still not satisfactory due to the number of steps and the need to prepare the phenyl carbamate derivative and separate the phenol side product after the coupling step. Also, aminopyridines usually need to be made in several steps, for example via oxidation of a pyridine (II) and halogenation of the corresponding pyridine N-oxide (III) to give a halopyridine (IV) (X═F, Cl, Br) and substitution with ammonia or an ammonia derivative.

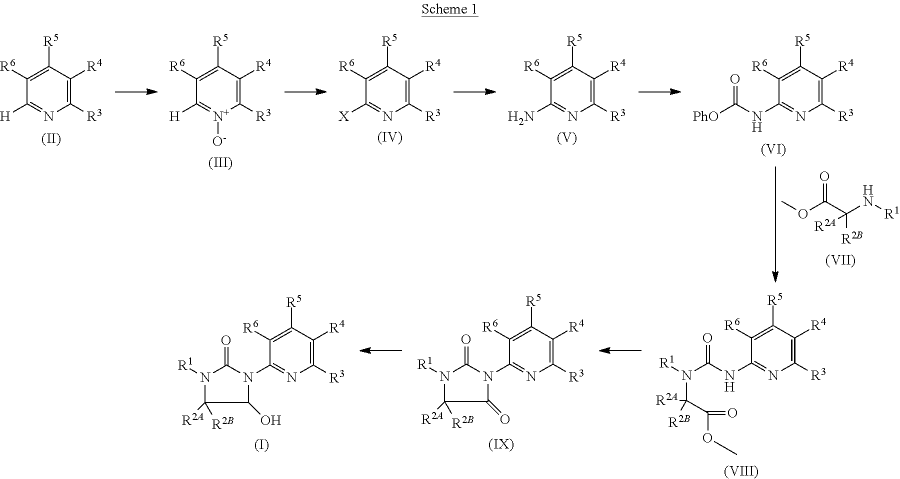

A very attractive approach toward the compounds of formula (I) would comprise an arylation of the hydantoin moiety in the 3-position providing directly the intermediates of formula (IX). However arylations of hydantoins in the 3-position are described as very difficult due to the low nucleophilicity of the hydantoin moiety. Typically, a high temperature and the presence of transition metal catalysts are needed (EP 436426, WO 2015100613, WO 2010029119). In many cases, product yields are disappointingly low and stoichiometric arylbismut, arylboron or aryllead derivatives have to be employed as the arylation reagents (Synlett 2006, 14, 2290-2292, J. Org. Chem. 1996, 61, 5865-5870). This approach is not satisfactory for large scale manufacturing and there is a great need for alternative hydantoin arylation methods. It is therefore the object of the present invention to provide a short and scalable process for the preparation of compounds of formula (I) via the intermediacy of an arylated hydantoin compound of formula (IX).

Surprisingly, it has now been found that compounds of formula (X) can be directly coupled with compounds of formula (III) in the presence of an activating agent and base—a hitherto unknown coupling of hydantoins with pyridine-N-oxides. Compounds of formula (IX) can then be reduced to compounds of formula (I) (Scheme 2). This invention therefore provides a one-step process for the production of compounds of formula (IX) and, in addition, a short and scalable process for the preparation of herbicidally active compounds of formula (I).

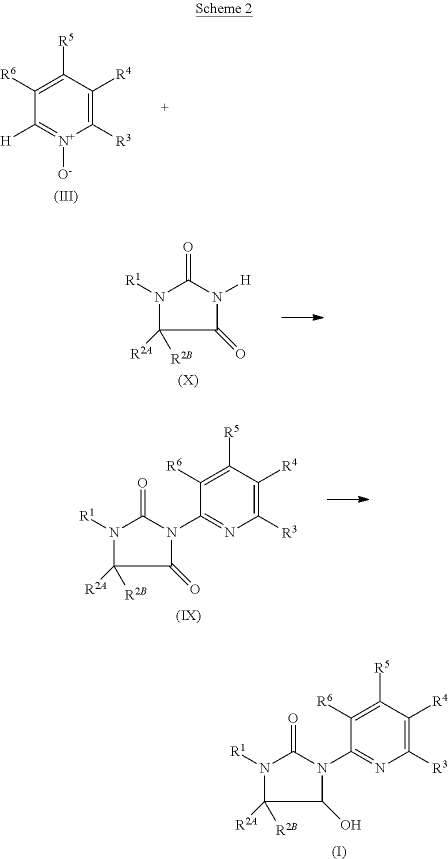

Thus, according to the present invention, there is provided a process for the preparation of compound of formula (IX)

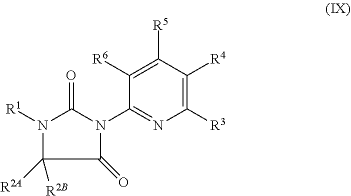

wherein

R¹ is selected from C₁-C₆ alkyl, aryl and hydrogen;

R^{2A} is selected from C₁-C₆ alkyl and hydrogen;

R^{2B} is selected from C₁-C₆ alkyl and hydrogen;

or R¹ and R^{2A} or R^{2B}, together with the nitrogen and carbon atoms to which they are attached form a 3-7 membered saturated ring optionally comprising from 1 to 3 heteroatoms independently selected from S, O and N and optionally substituted with from 1 to 3 groups independently selected from hydroxyl, ═O, C₁-C₆ alkyl and C₁-C₆ haloalkyl and

R³, R⁴, R⁵ and R⁶ are independently selected from hydrogen, C₁-C₆ alkyl, C₁-C₆ haloalkyl, nitro and halogen;

comprising reacting the compound of formula (III)

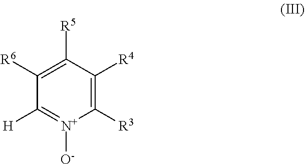

wherein R³, R⁴, R⁵ and R⁶ are as defined above with the compound of formula (X)

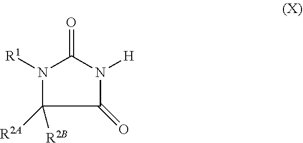

wherein R¹, R^{2A} and R^{2B} are as defined above in the presence of an activating agent and a base to form a compound of formula (IX)

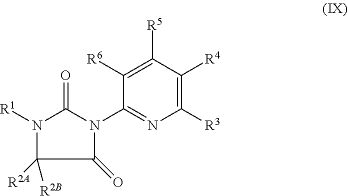

wherein R¹, R^{2A}, R^{2B}, R³, R⁴, R⁵ and R⁶ are as defined above.

Conveniently, the compound of formula (I) is then produced by reducing the compound of formula (IX).

In particularly preferred embodiments of the invention, preferred groups for R¹, R^{2A}, R^{2B}, R³, R⁴, R⁵ and R⁶, in any combination thereof, are as set out below.

Preferably, R¹ is selected from hydrogen and C₁-C₄ alkyl or R¹ and R^{2A} or R^{2B} form the group —CH₂CH₂CH₂CH₂—. More preferably, R¹ is selected from hydrogen and methyl and more preferably, methyl.

Preferably R² is selected from hydrogen and C₁-C₄ alkyl or R¹ and R^{2A} form the group —CH₂CH₂CH₂CH₂—. More preferably, R^{2A} is selected from hydrogen and methyl and, more preferably, hydrogen.

Preferably R^{a} is selected from hydrogen and C₁-C₄ alkyl or R¹ and R^{2B} form the group —CH₂CH₂CH₂CH₂—. More preferably, R^{2B} is selected from hydrogen and methyl and, more preferably, hydrogen.

Preferably R³ is selected from hydrogen, C₁-C₄ haloalkyl and halo. More preferably, R³ is selected from hydrogen, chloro, difluoromethyl and trifluoromethyl. More preferably, R³ is hydrogen.

Preferably R⁴ is selected from hydrogen, C₁-C₄ haloalkyl and halo. More preferably, R⁴ is selected from hydrogen, chloro, difluoromethyl and trifluoromethyl. More preferably, R⁴ is selected from hydrogen and trifluoromethyl and, more preferably, hydrogen.

Preferably R⁵ is selected from hydrogen, C₁-C₄ haloalkyl and halo. More preferably, R⁵ is selected from hydrogen, chloro, difluoromethyl and trifluoromethyl. More preferably, R⁵ is selected from hydrogen, trifluoromethyl and chloro. More preferably, R⁵ is trifluoromethyl.

Preferably R⁶ is selected from hydrogen, C₁-C₄ haloalkyl and halo. More preferably, R⁶ is selected from hydrogen, chloro, difluoromethyl and trifluoromethyl. More preferably, R⁶ is hydrogen.

The following scheme 3 describes the reactions of the invention in more detail. The substituent definitions are the same as defined above. The starting materials as well as the intermediates may be purified before use in the next step by state of the art methodologies such as chromatography, crystallization, distillation and filtration.

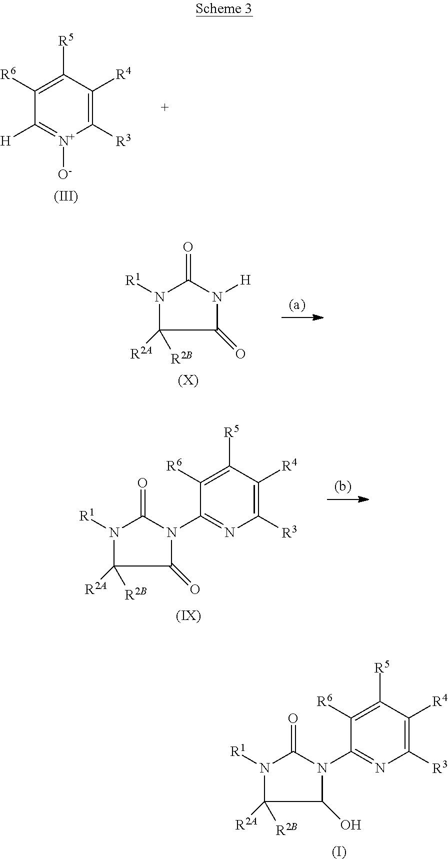

### Step (a):

The compound of formula (IX) can be advantageously prepared by reacting a compound of formula (III) with compound of formula (X) in presence of an activating agent and a base.

Suitable bases include, but are not limited to, trialkyl amines, alkali metal carbonates, alkali metal hydrogenocarbonates, pyridine derivatives, dialkylaniline derivatives and alkali metal salts of derivative (X). Particularly preferred are trialkyl amines and alkali metal carbonates such as diisopropylethylamine, triethylamine, tripropylamine, tributyl amine sodium carbonate and potassium carbonate. More preferably, the base is diisopropylethylamine. The amount of the base is typically between 1 and 20 equivalents, more preferably between 1 and 10 equivalents.

Suitable activating agents include, but are not limited to chloroformates, carbamoylchlorides, sulphonic acid chlorides, sulphonic acid anhydrides, chlorophosphates, phosphoric acid anhydrides, carboxylic acid anhydrides and carboxylic acid chlorides. Preferred activation agents are chloroformates, sulphonic acid chlorides and sulphonic acid anhydrides. Particularly preferred activation agents are chloroformates such as methylchloroformate, ethylchloroformate, propylchloroformate, iso-propylchloroformate, butylchloroformate andphenylchloroformate. The most preferred activating agents are methylchloroformate and ethylchloroformate and, more preferably, methylchloroformate. The amount of activating agents is typically between 1.00 and 10.00 equivalents, more preferably between 1 and 5 equivalents.

The reactions between compounds of formula (III) and (X) are preferably carried out in the presence of a solvent. Suitable solvents include, but are not limited to non-protic organic solvents such as tetrahydrofuran, 2-methyl tetrahydrofuran, toluene, xylenes, chlorobenzene, dichloromethane, 1,2-dichloroethane, dioxane, acetonitrile, ethylacetate.

Preferred solvents are acetonitrile, dichloromethane and tetrahydrofuran and, more preferably, acetonitrile.

The reactions between compounds of formula (III) and (X) are preferably carried by addition of the activation agent to a mixture of the compounds of formula (III) and (X) and the base.

The reaction can be carried out at a temperature from 0° C. to 150° C., preferably from 20° C. to 100° C. and most preferably from 50° C. to 100° C. (e.g. no lower than 0° C., preferably no lower than 20° C. and more preferably no lower than 50° C.; e.g. no more than 150° C., preferably no more than 100° C.).

Pyridine N-oxides (III), where not commercially available, may be made by literature routes such as below and as detailed in J. March, Advanced Organic Chemistry, 4^{th} ed. Wiley, New York 1992:

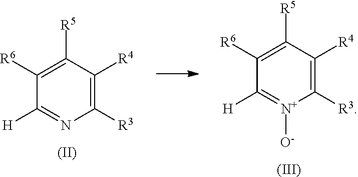

Suitable conditions for effecting these transformations are set out in J. March, Advanced Organic Chemistry, 4^{th} ed. Wiley, New York 1992.

Hydantoins (X), where not commercially available, may be made by literature routes such as below and as detailed in Chem. Rev., 1950, 46, 403-470:

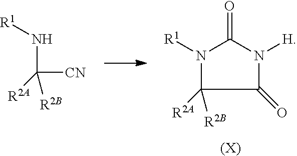

Suitable conditions for effecting these transformations are set in Chem. Rev., 1950, 46, 403-470.

### Step (b):

The compound of formula (I) can be advantageously prepared by reacting a compound of formula (IX) with a reducing agent. In principle any reducing agent known to a person skilled in the art for selective reduction of hydantoin structure could be employed. Suitable reducing agents include, but are not limited to borohydrides, aluminiumhydrides, boranes, metals, metal hydrides, silanes in the presence of a catalyst, hydrogen in the presence of a catalyst and formic acid in the presence of a catalyst. Suitable catalysts are known to the person skilled in the art. Preferred reducing agents are DIBAL-H, borane, NaBH₄, LiBH₄, KBH₄, LiAlH₄, polymethyihydrosiloxane, phenylsilane, sodium bis(2-methoxyethoxy)aluminumhydride and tetramethyldisiloxane. Some of the most preferred reagents include but are not limited to NaBH₄ and DIBAL-H and, more preferably, NaBH₄.

The amount of reducing agent is typically between 0.25 and 4.0 equivalents. For example, the amount of NaBH₄ is typically between 0.25 and 3.00 equivalents, more preferably between 0.3 and 1.5 equivalents. The amount of DIBAL-H is between 1.0 and 4.0 equivalents, more preferably between 1.0 and 2.0 eq.

The reduction of compound (IX) to compound (I) is preferably carried out in the presence of a solvent. Suitable solvents are protic or aprotic solvents or a mixture of the two. Suitable protic solvents are methanol, ethanol, isopropanol and water. Suitable aprotic solvents are tetrahydrofuran, 2-methyl tetrahydrofuran, 1,2-dichloroethane, 1,2-dimethoxyethane, chlorobenzene, dichlorobenzene, xylene and toluene. The selection of solvents will be dependent on the reducing agent. For example, when the reducing agent is NaBH₄, suitable solvents include, but are not limited to protic solvents and mixtures of protic and aprotic solvents such as methanol, ethanol, water, water/THF mixtures and methanol/THF mixtures and, more preferably, the solvent is a methanol/THF mixture; when the reducing agent is DIBAL-H, suitable solvents include, but are not limited to, aprotic solvents such as tetrahydrofuran, 2-methyl tetrahydrofuran, 1,2-dichloroethane and toluene.

The reaction can be carried out at a temperature from −20° C. to 100° C., preferably from −10° C. to 30° C. (e.g. no lower than −20° C. preferably no lower than −10° C.; e.g. no more than 100° C., preferably no more than 30° C.).

A number of specific intermediates of formula (IX) are novel. As such, the present invention also provides a novel intermediate of formula (IX) selected from the group consisting of:

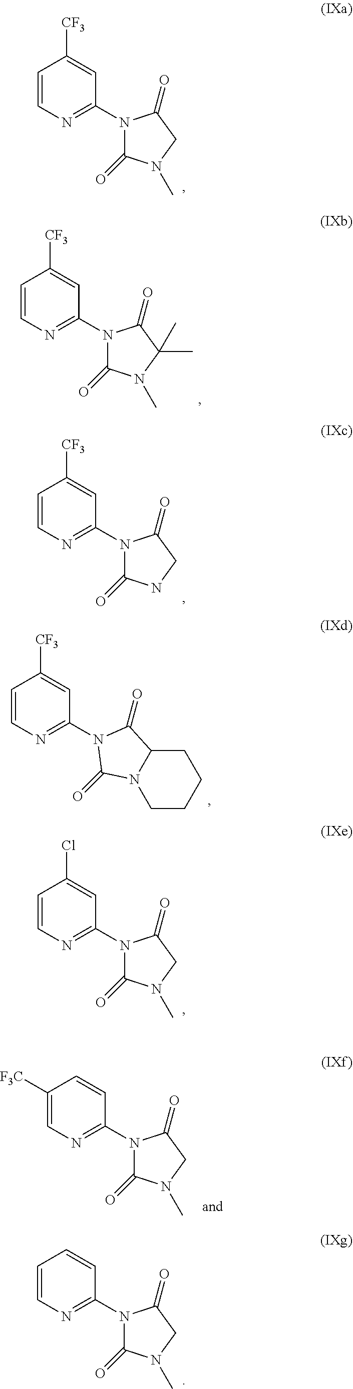

The compounds used in the process of the invention may exist as different geometric isomers, or in different tautomeric forms. This invention covers the production of all such isomers and tautomers, and mixtures thereof in all proportions, as well as isotopic forms such as deuterated compounds.

The compounds used in the process of this invention may also contain one or more asymmetric centers and may thus give rise to optical isomers and diastereomers. While shown without respect to stereochemistry, the present invention includes all such optical isomers and diastereomers as well as the racemic and resolved, enantiomerically pure R and S stereoisomers and other mixtures of the R and S stereoisomers and agrochemically acceptable salts thereof. It is recognized certain optical isomers or diastereomers may have favorable properties over the other. Thus when disclosing and claiming the invention, when a racemic mixture is disclosed, it is clearly contemplated that both optical isomers, including diastereomers, substantially free of the other, are disclosed and claimed as well.

Alkyl, as used herein, refers to an aliphatic hydrocarbon chain and includes straight and branched chains e. g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

Halogen, halide and halo, as used herein, refer to iodine, bromine, chlorine and fluorine.

Haloalkyl, as used herein, refers to an alkyl group as defined above wherein at least one hydrogen atom has been replaced with a halogen atom as defined above. Preferred haloalkyl groups are dihaloalkyl and trihaloalkyl groups. Examples of haloalkyl groups include chloromethyl, dichloromethyl, trichloromethyl, fluoromethyl, difluoromethyl and trifluoromethyl. Preferred haloalkyl groups are fluoroalkyl groups, especially difiluoroalkyl and trifluoroalkyl groups, for example, difluoromethyl and trifluoromethyl.

Nitro, as used herein, refers to the group —NO₂.

Aryl, as used herein, refers to an unsaturated aromatic carbocyclic group of from 6 to 10 carbon atoms having a single ring (e. g., phenyl) or multiple condensed (fused) rings, at least one of which is aromatic (e.g., indanyl, naphthyl). Preferred aryl groups include phenyl, naphthyl and the like. Most preferably, an aryl group is a phenyl group.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

The following abbreviations were used in this section: s=singlet; bs=broad singlet; d=doublet; dd=double doublet; dt=double triplet; t=triplet, tt=triple triplet, q=quartet, sept=septet; m=multiplet; RT=retention time, MH⁺=molecular mass of the molecular cation.

¹H and ¹⁹F NMR spectra were recorded on a Bruker Avance III 400 spectrometer equipped with a BBFOplus probe at 400 MHz/376.6 MHz, respectively.

### Example 1: Preparation of 1-methyl-3-[4-(trifluoromethyl)-2-pyridyl]imidazolidine-2,4-dione (IXa)

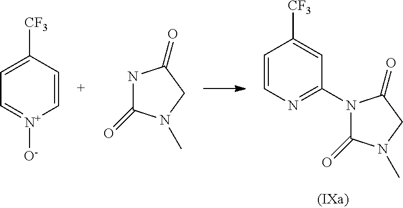

4-Trifluoropyridine-N-oxide (10.00 g), 1-methylimidazolidine-2,4-dione (7.62 g) and diisopropyl-ethyl amine (11.53 g) were mixed with dry acetonitrile (105 mL) under argon. The reaction mixture was warmed to 55° C. (internal temperature) and stirred at this temperature for 15 minutes. Methylchloroformate (8.51 g) was added slowly (addition rate=0.3 mL/min) resulting in temperature rise up to 63° C. After addition, the reaction mixture was stirred for additional 60 minutes at 60° C. After cooling to room temperature the reaction solvent was removed by evaporation. The crude product was diluted with dichloromethane, extracted with sodium carbonate solution (2×), 2M HCl (2×) and brine. The organic layer was dried over Na₂SO₄ and concentrated to give 15.7 g of (IXa) (86% purity determined by ¹H NMR) as a light yellow solid. Analytically pure sample was obtained by crystallization of the product from methanol-water (3:7).

¹H NMR (CDCl₃) δ=8.82 (d, J=6.7 Hz, 1H), 7.67 (s, 1H), 7.56 (d, J=6.7 Hz, 1H), 4.09 (s, 2H), 3.10 (s, 3H) ppm

¹⁹F NMR (CDCl₃) δ=−64.7 ppm

### Example 2: Preparation of 4-hydroxy-1-methyl-3-[4-(trifluoromethyl)-2-pyridyl]imidazolidin-2-one

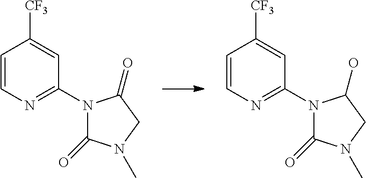 [SMILES: CN1CC(=O)N(c2cc(C(F)(F)F)ccn2)C1=O ; CN1CC(O)N(c2cc(C(F)(F)F)ccn2)C1=O]

1-methyl-3-[4-(trifluoromethyl)-2-pyridyl]imidazolidine-2,4-dione (IXa) (1.00 g) was mixed with dry tetrahydrofurane (9.0 mL) and methanol (1 mL) under argon. The reaction mixture was cooled to 0° C. (internal temperature) and then sodium borohydride (83 mg) was added portion wise over a period of 15 minutes. The reaction mixture was stirred at 0-5° C. for 2 h. The reaction mass was concentrated under reduced pressure to half the volume. Then the reaction mass was diluted with water (10 mL) and a white solid precipitated. The solid was filtered and dried in high vacuum providing the 0.85 g of 4-hydroxy-1-methyl-3-[4-(trifluoromethyl)-2-pyridyl]imidazolidin-2-one as a white solid (93% purity determined by ¹H NMR)

Analytical data matches those reported in WO 2015/059262

### Example 3: preparation of 1,5-dimethyl-3-[4-(trifluoromethyl)-2-pyridyl]imidazolidine-2,4-dione

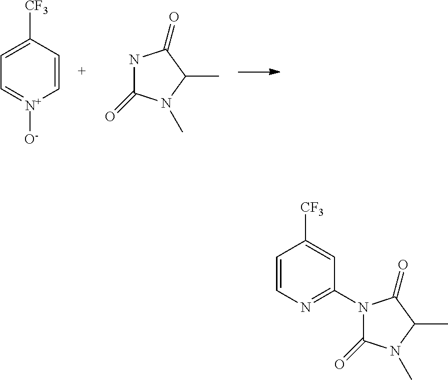

4-Trifluoropyridine-N-oxide (1.00 g), 1,5-dimethylmethylimidazolidine-2,4-dione (0.864 g) and diisopropyl-ethyl amine (1.19 g) were mixed with dry acetonitrile (10 mL) under argon. The reaction mixture was warmed to 55° C. (internal temperature) and stirred at this temperature for 15 minutes. Methylchloroformate (0.88 g) was added during 30 minutes at 60° C. After addition, the reaction mixture was stirred for additional 60 minutes at 60° C. After cooling to room temperature the reaction solvent was removed by evaporation. The crude product was diluted with dichloromethane, extracted with sodium carbonate solution (2×), 2M HCl (2×) and brine. The organic layer was dried over Na₂SO₄ and concentrated. The crude product was purified by column chromatography (silica, cyclohexane/ethylacetate gradient) providing 1,5-dimethyl-3-[4-(trifluoromethyl)-2-pyridyl]imidazolidine-2,4-dione as an off white solid 1.02 g.

Analytical data matches those reported in WO 2015/052076

Reduction to the compound of formula (I) is described in WO 2015/052076.

### Example 4: Preparation of 1,5,5-trimethyl-3-[4-(trifluoromethyl)-2-pyridyl]imidazolidine-2,4-dione (IXb)

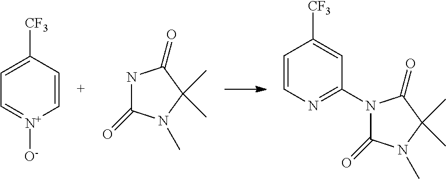

4-Trifluoropyridine-N-oxide (1.00 g), 1,5,5-trimethylimidazolidine-2,4-dione (0.959 g) and diisopropyl-ethyl amine (4.6 mL) were mixed with dry acetonitrile (10 mL) under argon. The reaction mixture was warmed to 55° C. (internal temperature) and stirred at this temperature for 15 minutes. Methylchloroformate (0.88 g) was added during 30 minutes at 60° C. After addition, the reaction mixture was stirred for additional 2 h at 60° C. After cooling to room temperature, the reaction solvent was removed by evaporation. The crude product was diluted with dichloromethane, extracted with sodium carbonate solution (2×), 2M HCl (2×) and brine. The organic layer was dried over magnesium sulphate and concentrated. The crude product was purified by column chromatography (silica, cyclohexane/ethylacetate gradient) providing 1.14 g of 1,5,5-trimethyl-3-[4-(trifluoromethyl)-2-pyridyl]imidazolidine-2,4-dione as a white solid.

¹H NMR (CDCl₃) δ=8.80 (d, J=5.1 Hz, 1H), 7.68 (s, 1H), 7.5 (d, J=5.1 Hz, 1H), 2.97 (s, 3H), 1.52 (s, 6H) ppm

¹⁹F NMR (CDCl₃) δ=−64.7 ppm

Table 1 lists compounds of the general formula

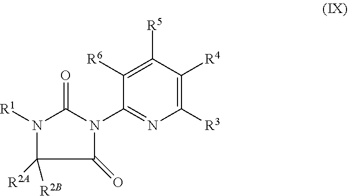

wherein R¹, R^{2A}, R^{2B}, R³, R⁴, R⁵ and R⁶ are as defined in the table.

These compounds were made by the general methods of Examples 1 to 4.

TABLE 1

| | | |
|---|---|---|
| 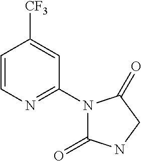 [SMILES: O=C1CNC(=O)N1c1cc(C(F)(F)F)ccn1] | (IXc) | ¹H NMR (CDCl₃) δ = 8.86 (d, J = 4.8 Hz, 1H), 7.7 (s, 1H), 7.6 (d, J = 4.8 Hz, 1H), 5.7 (br s, 1H), 4.22 (s, 2H) ¹⁹F NMR (CDCl₃) δ = −64.6 ppm |
| 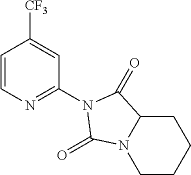 [SMILES: O=C1C2CCCCN2C(=O)N1c1cc(C(F)(F)F)ccn1] | (IXd) | ¹H NMR (CDCl₃) δ 8.82 (d, J = 5.1 Hz, 1H), 7.69 (s, 1H), 7.54 (d, J = 5.1 Hz, 1H), 4.27 (m, 1H), 3.97 (m, 1H), 2.93 (m, 1H), 2.32 (m, 1H), 2.07 (m, 1H), 1.81 (d, J = 12.8 Hz, 1H), 1.47-1.59 (m, 3H) ¹⁹F NMR (CDCl₃) δ = −64.7 ppm |
| 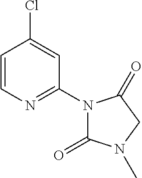 | (IXe) | ¹H NMR (CDCl₃) δ 8.55 (d, J = 5.1 Hz, 1H), 7.46 (d, J = 1.8 Hz, 1H), 7.35 (dd, J = 5.1 Hz, J = 1.8 Hz, 1H), 4.07 (s, 2H), 3.1 (s, 3H) |
| 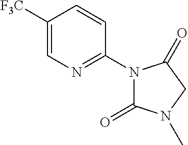 [SMILES: CN1CC(=O)N(c2ccc(C(F)(F)F)cn2)C1=O] | (IXf) | ¹H NMR (CDCl₃) δ = 8.91 (s, 1H), 8.09-8.11 (m, 1H), 7.60 (d, J = 8.1 Hz, 1H), 4.11 (s, 2H), 3.12 (s, 3H) ppm ¹⁹F NMR (CDCl₃) δ = −62.4 ppm |
| 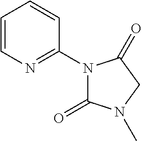 | (IXg) | ¹H NMR (CDCl₃) δ = 8.64 (m, 1H), 7.86 (m, 1H), 7.33-7.40 (m, 2H), 4.07 (s, 2H), 3.09 (s, 3H) ppm |

1. A process for the preparation of compound of formula (IX) 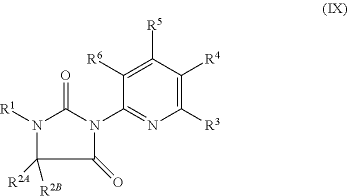
wherein
R¹ is selected from C₁-C₆ alkyl, aryl and hydrogen;
R^{2A} is selected from C₁-C₆ alkyl and hydrogen;
R^{2B} is selected from C₁-C₆ alkyl and hydrogen;
or R¹ and R^{2A} or R^{2B}, together with the nitrogen and carbon atoms to which they are attached form a 3-7 membered saturated ring optionally comprising from 1 to 3 heteroatoms independently selected from S, O and N and optionally substituted with from 1 to 3 groups independently selected from hydroxyl, ═O, C₁-C₆ alkyl and C₁-C₆ haloalkyl and
R³, R⁴, R⁵ and R⁶ are independently selected from hydrogen, C₁-C₆ alkyl, C₁-C₆ haloalkyl, nitro and halogen;
comprising reacting the compound of formula (III) 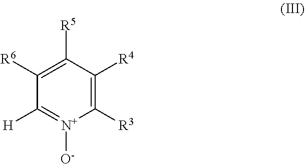
wherein R³, R⁴, R⁵ and R⁶ are as defined above with the compound of formula (X) 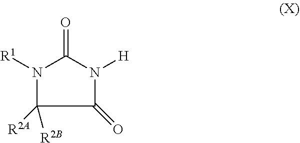
wherein R¹, R^{2A} and R^{2B} are as defined above in the presence of an activating agent and a base to form a compound of formula (IX) 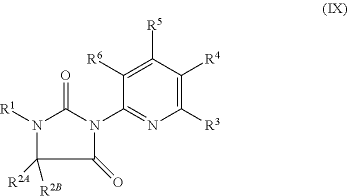
wherein R¹, R^{2A}, R^{2B}, R³, R⁴, R⁵ and R⁶ are as defined above.

2. The process of claim 1, wherein the base is selected from the group consisting of trialkyl amines, alkali metal carbonates, alkali metal hydrogenocarbonates, pyridine derivatives, dialkylaniline derivatives and alkali metal salts of derivative (X).

3. The process of claim 1, wherein the activating agent is selected from the group consisting of chloroformates, carbamoylchlorides, sulphonic acid chlorides, sulphonic acid anhydrides, chlorophosphates, phosphoric acid anhydrides, carboxylic acid anhydrides and carboxylic acid chlorides.

4. The process of claim 1, which is carried out in the presence of a solvent.

5. The process of claim 1, which is carried out by addition of the activation agent to a mixture of the compounds of formula (III) and (X) and the base.

6. The process of claim 1, which is carried out at a temperature from 0° C. to 150° C.

7. A process for the preparation of a compound of formula (I): 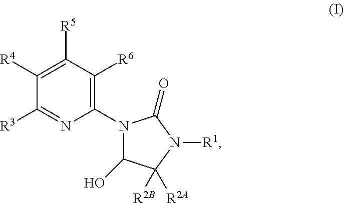
comprising the preparation of a compound of formula (IX) according to claim 1 followed by reduction of the compound of formula (IX) in the presence of a reducing agent to the compound of formula (I):

8. The process of claim 7, wherein the reducing agent is selected from the group consisting of borohydrides, aluminium hydrides, boranes, metals, metal hydrides, silanes in the presence of a catalyst, hydrogen in the presence of a catalyst and formic acid in the presence of a catalyst.

9. The process of claim 7, wherein the reaction is carried out at a temperature from −20° C. to 100° C.

10. The process of claim 1, wherein R¹ is selected from hydrogen and C₁-C₄ alkyl or R¹ and R^{2A} or R^{2B} form the group —CH₂CH₂CH₂CH₂—.

11. The process of claim 10, wherein R¹ is selected from hydrogen and methyl.

12. The process of claim 1, wherein R^{2A} is selected from hydrogen and C₁-C₄ alkyl or R¹ and R^{2A} form the group —CH₂CH₂CH₂CH₂—

13. The process of claim 12, wherein R^{2A} is selected from hydrogen and methyl.

14. The process of claim 1, wherein R^{2B} is selected from hydrogen and C₁-C₄ alkyl or R¹ and R^{2B} form the group —CH₂CH₂CH₂CH₂—

15. The process of claim 14, wherein R^{2B} is selected from hydrogen and methyl.

16. The process of claim 1, wherein R³ is selected from hydrogen, C₁-C₄ haloalkyl and halo.

17. The process of claim 16, wherein R³ is selected from hydrogen, chloro, difluoromethyl and trifluoromethyl.

18. The process of claim 1, wherein R⁴ is selected from hydrogen, C₁-C₄ haloalkyl and halo.

19. The process of claim 18, wherein R⁴ is selected from hydrogen, chloro, difluoromethyl and trifluoromethyl.

20. The process of claim 1, wherein R⁵ is selected from hydrogen, C₁-C₄ haloalkyl and halo.

21. The process of claim 20, wherein R⁵ is selected from hydrogen, chloro, difluoromethyl and trifluoromethyl.

22. The process of claim 1, wherein R⁶ is selected from hydrogen, C₁-C₄ haloalkyl and halo.

23. The process of claim 22, wherein R⁶ is selected from hydrogen, chloro, difluoromethyl and trifluoromethyl.

24. A compound of formula (IX) selected from the group consisting of: [chemical structure]
